(19) United States
(12) Patent Application Publication
(10) Pub. No.: US 2010/0036323 A1
(43) Pub. Date: Feb. 11, 2010

(54) FLEXIBLE CANNULA WITH SEAL

(76) Inventor: Robert C. Smith, Middletown, CT (US)

Correspondence Address: Tyco Healthcare Group LP, 60 MIDDLETOWN AVENUE, NORTH HAVEN, CT, 06473, US

(21) Appl. No.: 12/525,608

(22) PCT Filed: Feb. 15, 2008
(86) PCT No.: PCT/US08/02072

§ 371 Date: Aug. 3, 2009

Related U.S. Application Data
(60) Provisional application No. 60/902,416, filed on Feb. 20, 2007.

(51) Int. Cl. A61B 17/34

(52) U.S. Cl. 604/167.01

(57) ABSTRACT

A flexible cannula assembly includes a tubular member having a proximal end portion and a distal end portion and a seal positioned adjacent the distal end portion of the tubular member. The tubular member is formed of a flexible material. The seal defines a hole in a central portion thereof for receiving surgical instruments therethrough. The flexible cannula assembly further includes a housing connected to the proximal end portion of the tubular member.

### BACKGROUND

1. Field of the Disclosure

The present disclosure relates to a flexible cannula including a sealing device. More particularly, the present disclosure relates to a cannula for use in laparoscopic surgical procedures including a sealing member which seals the cannula lumen upon insertion of an instrument through the cannula.

2. Background of Related Art

Surgical procedures have been developed during which surgical instruments are passed through small openings in body tissue to access internal surgical sites. These surgical procedures, commonly referred to as endoscopic procedures, have become widely accepted. The term endoscopic as used herein is defined to include all types of minimally invasive surgical procedures including laparoscopic and arthroscopic procedures. Typically, during these procedures, after an incision has been formed in the body tissue, a cannula defining a lumen is inserted through the incision and fixedly positioned in relation to the surgical site. During some such procedures, the body cavity is inflated with an insufflation gas to create a working area inside a patient and allow a trocar to penetrate a body cavity without the risk of damaging underlying organs within the body cavity.

It is important in endoscopic procedures to minimize undesired fluid flow to and from the surgical site; and, accordingly, the cannula must be sealed prior to and subsequent to the introduction of surgical instruments and while such instruments are in place. In addition, fluids, such as gaseous phase carbon dioxide or nitrous oxide, may be introduced into the anatomical cavity for insufflation as part of the endoscopic procedure, and the escape of such fluids must be minimized during penetration of the cavity as well as during the operative procedure. The valves of endoscopic portals, such as cannulas, typically have a valve passage with a size corresponding to an outer diameter or size of the penetrating instrument to form a seal with the penetrating instrument, the size of the penetrating instrument varying in accordance with the endoscopic procedure being performed and the type of anatomical cavity being penetrated.

Generally, endoscopic cannulas include a sealing member or members to seal the cannula lumen prior to and after insertion of a surgical instrument into the body cavity to prevent insufflation gases within the body cavity from escaping. The sealing member or members often include adjustable sealing elements capable of sealing about multiple instruments of different sizes and shapes. Many prior art cannulas utilize a flapper or gate valve that is normally biased to a closed position and movable to an open position to allow the penetrating instrument to be inserted through the valve passage, which has a single, predetermined size corresponding to the size of the penetrating instrument.

Although flexible cannulas having seals which adequately perform the intended functions are known, improvements to the known devices are warranted. For example, it is common practice to have a sealing member mounted in a proximal housing portion of the cannula. Generally, this configuration adequately performs the intended function of preventing the escape of insufflation gases. However, having the sealing member positioned in the proximal housing portion of the cannula causes the housing dimensions to be relatively large.

Additionally, in view of the widespread acceptance of endoscopic procedures in surgery, numerous endoscopic instruments have been developed which allow the surgeon to perform complex surgical procedures with minimal incision into the skin and tissue surrounding a particular body cavity or anatomical region. Many of these endoscopic instruments are rigid and in a fixed position, thus inhibiting the surgeon's ability to maneuver the instrument within the cavity.

Although an increasing number of endoscopic instruments incorporate the ability to flex during use, the sealing member remains in the proximal portion of the cannula resulting in a cannula assembly which is relatively large. Thus, a continuing need exists for a flexible self-sealing cannula which is compact in size and prevents the loss of insufflation gas while providing a user with the ability, and maneuverability, to perform endoscopic procedures with surgical instruments positioned within the flexible self-sealing cannula.

### SUMMARY

The present disclosure provides a novel flexible self-sealing cannula which is compact in size and prevents the loss of insufflation gas while providing a user with the maneuverability to perform endoscopic procedures with surgical instruments positioned within the flexible self-sealing cannula.

The flexible self-sealing cannula assembly includes a flexible cannula having an instrument seal on a distal end thereof The flexibility of the cannula and seal permit surgical personnel to maneuver surgical instruments within the cannula during endoscopic procedures, without compromising the integrity of the seal.

In accordance with an embodiment of the present disclosure, a flexible cannula is provided which includes a tubular member having a proximal end portion and a distal end portion and an instrument seal positioned adjacent the distal end portion of the tubular member. The seal defines a hole in a central portion thereof for receiving surgical instruments therethrough. The cannula is formed of a resilient flexible material. The flexibility of the cannula, combined with the fact that the seal is positioned adjacent a distal end of the cannula, provides the surgical personnel with maneuverability advantages when utilizing the apparatus.

The flexible cannula also includes a housing positioned adjacent and proximal to the proximal end portion of the tubular member. A zero-seal is preferably mounted within the housing. A guide member is attached to a distal end of the tubular member to facilitate easy insertion of the flexible cannula into the patient.

In another embodiment of the present disclosure, a flexible, self-sealing cannula assembly is provided including a cannula body having a proximal end portion and a distal end portion, wherein the flexible cannula includes an instrument seal mounted on a distal portion thereof. The cannula body defines a longitudinal lumen for removably receiving surgical instruments therein. The proximal end portion of the cannula body defines an inlet opening and the distal end portion defines an outlet opening.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiments given below, serve to explain the principles of the disclosure.

FIG. 1 is a side cross-sectional view of a self-sealing cannula assembly in accordance with an embodiment of the present disclosure;

FIG. 2 is a perspective view of a self-sealing cannula in accordance with an embodiment of the present disclosure;

FIG. 3 is a partial perspective view of a self-sealing cannula in accordance with another embodiment of the present disclosure;

FIG. 4 is an exploded perspective view of a self-sealing cannula assembly in accordance with an embodiment of the present disclosure;

FIG. 5 is a perspective view of a self-sealing cannula in accordance with an embodiment of the present disclosure;

FIG. 6 is a side cross-sectional view of a self-sealing cannula assembly having an instrument inserted therein, in accordance with an embodiment of the present disclosure;

FIG. 7 is a side cross-sectional view of a self-sealing cannula assembly having an instrument inserted therein, in accordance with an embodiment of the present disclosure;

FIG. 8 is a side cross-sectional view of a flexible, self-sealing cannula assembly in accordance with an embodiment of the present disclosure; and

FIG. 9 is a side cross-sectional view of a tubular portion and seal of a flexible, self-sealing cannula assembly in accordance with another embodiment of the present disclosure.

### DETAILED DESCRIPTION OF PREFERRED EMBODIMENTS

Preferred embodiments of the presently disclosed self-sealing cannula will now be described in detail with reference to the figures, in which like reference numerals identify corresponding elements throughout the several views.

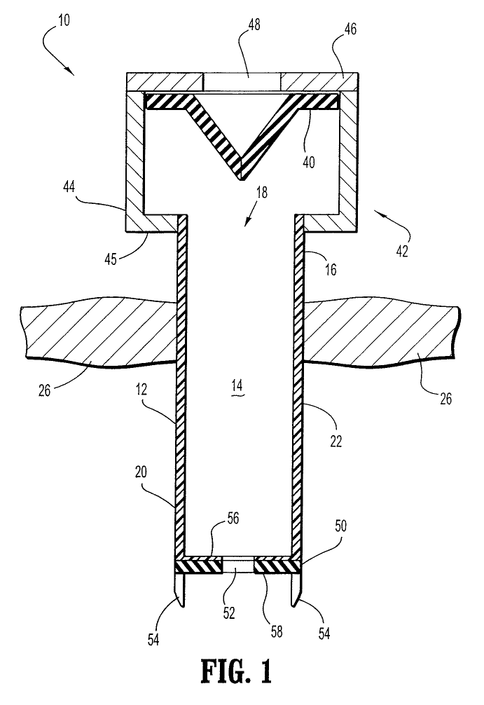

A self-sealing cannula assembly, shown generally as reference numeral 10 in FIG. 1, includes a tubular portion 12 which defines a longitudinal lumen 14. The tubular portion includes a proximal end portion 16, which preferably defines an inlet opening 18, a central body portion 22, which is preferably cylindrical, and a distal end portion 20.

Self-sealing cannula assembly 10 also includes a proximal housing portion 42. Proximal housing portion 42 includes a cylindrical member 44 and a cover 46. The cylindrical member has a shoulder 45 formed on the distal end thereof. The shoulder 45 defines an opening for receiving tubular portion 12. Proximal end portion 16 of the tubular portion 12 is secured in a manner that will form a seal between the proximal end portion 16 and shoulder 45. Cover 46 defines an inlet opening 48 for receiving surgical instruments into the self-sealing cannula assembly 10. Cover 46 engages the proximal end of cylindrical member 44 and may be attached thereto by methods known to one having ordinary skill in the art. Although self-sealing cannula assembly 10 is illustrated as being formed of multiple components, e.g., proximal housing portion 42 is formed separately from the tubular portion 12, it is envisioned that self-sealing cannula assembly 10, or any portion thereof, may be of monolithic construction.

An instrument seal 50 having a proximal surface 56 and a distal surface 58 is connected to the distal end portion 20 of the tubular portion 12. Instrument seal 50 defines an opening 52 in the central region of the seal. Since tubular portion 12 is flexible, surgical personnel will have the ability to maneuver the instruments within the cannula without compromising the effectiveness of seal 50.

The lumen 14 becomes pressurized by the insufflation gas which enters into the lumen 14 through the opening 52 in seal 50. The pressure against the inside surface of the flexible tubular portion 12 maintains a seal between the outer surface of flexible tubular portion 12 and the inner surface of the dermis 26 of the patient. Therefore, the loss of insufflation gas around the circumference of the cannula assembly is effectively prevented. An access port may be defined in tubular potion 12 or housing 42 to communicate directly with lumen 14 and to regulate the pressure within the lumen.

A flexible zero-seal 40 for sealing cannula lumen 14 to prevent or minimize the loss of gasses through inlet opening 48 is provided within proximal housing portion 42. Flexible zero-seal 40 is designed to provide a positive seal when there is no instrument positioned therein. Flexible zero-seal 40 may be a flexible membrane having an expandable slit formed therein. Alternately, other types of seals may be used in place of zero-seal 40, or one or more additional seals may be installed adjacent proximal housing portion 42.

In use, a body incision or hole is typically made through the dermis 26 of the patient with a trocar. The tubular portion 12 of self-sealing cannula assembly 10 is then positioned through the body incision into an insufflated cavity. The cavity may be pressurized with an insufflation medium through cannula assembly 10 or prior to insertion of cannula assembly 10. In accordance with an embodiment of this disclosure, at least one guide member 54 is attached to a distal end of flexible tubular portion 12 to facilitate easy entry of the cannula assembly 10 into the body incision. It is preferred that the distal end of guide member 54 is beveled as illustrated.

Once the cannula assembly is inserted into an insufflated cavity, pressurized gas from within the cavity flows into lumen 14 via opening 52 in seal 50, or via an access port (not shown), to inflate flexible tubular portion 12. Thereafter, when an instrument is inserted through lumen 14 and through instrument seal 50, restricting flow in or out of lumen 14, lumen 14 will remain pressurized. To ensure that the pressurized gas in the insufflated cavity does not escape upon the entry of an instrument through the lumen 14, flexible seal 40 will preferably seal uniformly around the body of the surgical instrument.

In one preferred embodiment, a synthetic material is used to form seal 40. Cannula assembly 10, according to the present disclosure, by virtue of seal 40, is primed for self-sealing when it is disposed in an insufflated body cavity. However, depending on, inter alia, materials of construction or configuration, it is possible that when a surgical instrument is inserted through lumen 14, seal 40 will not compress uniformly about the surgical instrument thereby creating a number of gaps between seal 40 and the surgical instrument. In prior art devices, these gaps would allow the pressurized gas in the insufflated cavity to escape from the body cavity, thereby minimizing the effectiveness of the self-sealing cannula. However, in accordance with the present disclosure, seal 40 is formed from a synthetic material that will compress uniformly around the body of a surgical instrument and form a pressure barrier which eliminates or minimizes the gaps. It is preferred that the synthetic material be nylon, Kevlar®, or any other material that will compress uniformly when a surgical instrument is inserted in the cannula assembly 10. The selected material may also be of knitted construction to minimize or prevent wrinkling of seal 40 when a surgical instrument is inserted into the cannula assembly. Notwithstanding the advances in the choice of materials and construction of the seal 40, it is likely that at least some insufflation gas will leak out. Accordingly, in accordance with the present disclosure, seal 50 assists in minimizing or eliminating the amount of gas that is bypassing seal 40.

The selected material will preferably have a low coefficient of friction so that insertion and removal of a surgical instrument does not require excessive amounts of force. An interior surface of cannula assembly 10 may also be coated with a lubricious material to minimize the friction between the surgical instrument and the tubular portion 12. Although cannula assembly 10 will preferably have a low coefficient of friction, the combination of seal 40 and seal 50 is capable of maintaining the surgical instrument properly positioned within cannula assembly 10 during the laparoscopic procedure. Further still, the selected material is preferably thin yet durable enough to prevent the surgical instrument from inadvertently puncturing any portion of the cannula assembly 10 during insertion, removal or operation of the surgical instrument.

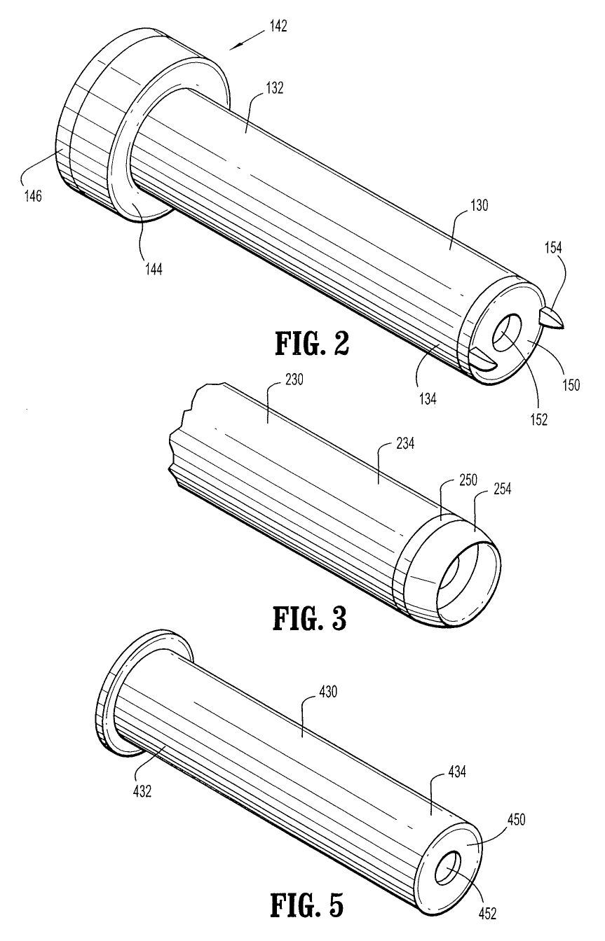

Referring now to FIG. 2, a perspective view of a flexible self-sealing cannula assembly 130 in accordance with an embodiment of the present disclosure is shown. Flexible self-sealing cannula 130 has a proximal end portion 132 and a distal end portion 134. A housing 142 is mounted on the proximal end portion 132 of flexible self-sealing cannula 130. Housing 142 includes a cylindrical member 144 and a cover 146. The cylindrical member 144 defines an opening for receiving cannula 130. The distal end portion 134 is inserted through the opening defined by cylindrical member 144. Cover 146 engages the proximal end of cylindrical member 144 and may be attached thereto by methods known to one having ordinary skill in the art.

A seal 150 is connected to the distal end portion 134 of the flexible cannula 130. Seal 150 defines an opening 152 in the central region of the seal. In accordance with the present disclosure, cannula 130 exhibits sufficient flexibility such that surgical personnel will have the ability to maneuver instruments within flexible cannula 130 without compromising the integrity and effectiveness of seal 150.

At least one guide member 154 is attached to a distal end of flexible cannula 130 to facilitate easy entry of the cannula into the body incision. It is preferred that the distal ends of guide members 154 are beveled as illustrated.

FIG. 3 is a partial perspective view of another embodiment of a tubular portion 230 of a self-sealing cannula assembly in accordance with the present disclosure. More specifically, FIG. 3 illustrates a partial view of a tubular portion of a cannula assembly having a guide member 254 attached to a distal end portion 234 of the tubular portion. As seen by comparing FIGS. 2 and 3, guide member 254 differs from guide members 154 most notably to the extent that guide member 254 is a full annular member. An instrument seal 250 is positioned at the distal end of the tubular portion 230 and adjacent to the guide member 254.

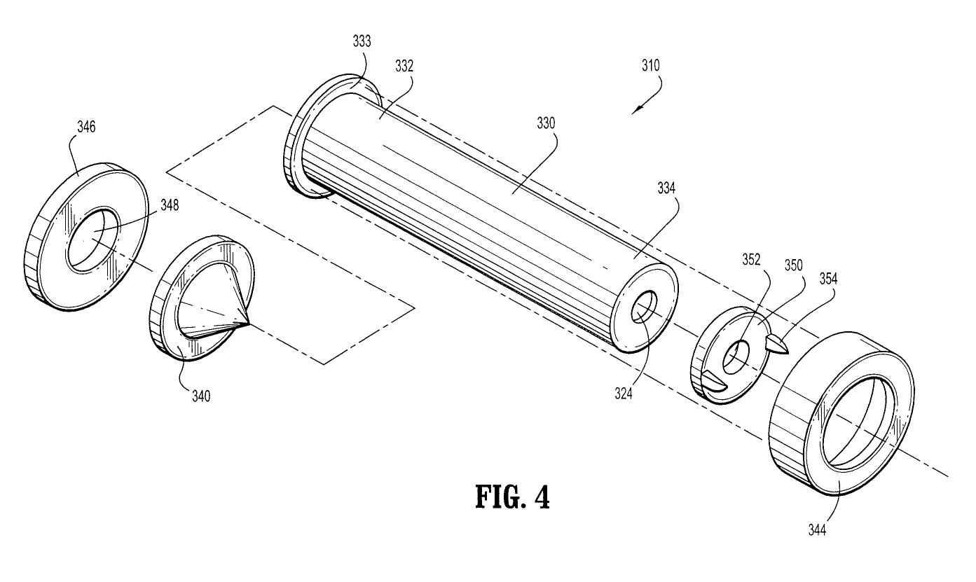

Referring now to FIG. 4, an exploded perspective view of the self-sealing cannula assembly illustrated in FIG. 1 is shown generally as reference numeral 310. Self-sealing cannula assembly 310 includes a tubular cannula body 330 which defines a longitudinal lumen (see FIG. 1). The tubular cannula body 330 includes a proximal end portion 332, a central body portion, which is preferably cylindrical, and a distal end portion 334, which defines an outlet opening 324.

A flexible zero-seal 340 is positioned adjacent to the proximal end portion 332 of cannula 330. Flexible zero-seal 340 seals uniformly around the body of a surgical instrument, to ensure that pressurized gas in an insufflated cavity does not escape through the lumen 314.

An instrument seal 350 having a proximal surface and a distal surface is connected to the distal end portion 334 of the flexible cannula 330. Instrument seal 350 defines an opening 352 in the central region of the seal for receiving surgical instruments which are inserted through flexible seal 340 and through cannula 330. Therefore, the combination of flexible seal 340 and instrument seal 350 is capable of maintaining the surgical instrument properly positioned within cannula assembly 310 during the laparoscopic procedure while minimizing or eliminating the loss of insufflation gas.

A guide member 354 is attached to a distal surface of instrument seal 350 to facilitate easy entry of the cannula assembly 310 into the body incision. It is preferred that the distal end of guide member 354 is beveled as illustrated.

Self-sealing cannula assembly 310 is held together by the components of a proximal housing portion. More specifically, the proximal housing portion includes a cylindrical member 344 and a cover 346. The cylindrical member 344 is configured to fit around the flexible tubular portion of cannula 330. Accordingly, cylindrical member 344 is slid in the proximal direction beginning at the distal end portion 334 of the cannula 330. The cylindrical member 344 is moved in the proximal direction until it engages a distal side of annular ring 333. At that point, the cover 346 engages the proximal end of cylindrical member 344 thereby forming the proximal housing portion. Self-sealing cannula assembly 310 is then ready to be inserted within a body incision or hole made through the dermis 326 of the patient with a trocar. Self-sealing cannula assembly 310 is then positioned through the body incision into an insufflated cavity.

Turning now to FIG. 5, a perspective view of a self-sealing cannula in accordance with another embodiment of the present disclosure is illustrated. More specifically, FIG. 5 illustrates a cannula 430 having a proximal end 432 and a distal end 434. The cannula 430 includes a seal 450 integrated into the distal end thereof. Seal 450 defines a circular opening 452 for receiving and sealing against a surgical instrument when the surgical instrument is inserted through the self-sealing cannula. It is contemplated that guide members, although not shown, may be attached to the distal end of cannula 430 as shown above.

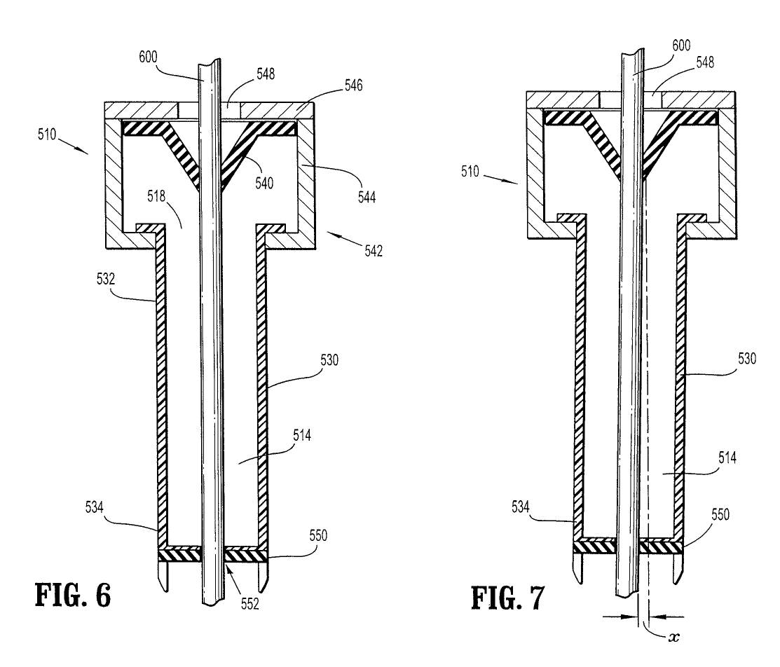

FIGS. 6 and 7 are side cross-sectional views of a flexible self-sealing cannula assembly 510 with a surgical instrument 600 inserted therein. Self-sealing cannula assembly 510 includes a cannula body 530 which defines a longitudinal lumen 514. The cannula body includes a proximal end portion 532, which preferably defines an inlet opening 518, and a distal end portion 534.

Self-sealing cannula assembly 510 also includes a proximal housing portion 542. Proximal housing portion 542 includes a cylindrical member 544 and a cover 546. Cover 546 defines an inlet opening 548 for receiving surgical instruments into the self-sealing cannula assembly 510.

An instrument seal 550 is connected to the distal end of the flexible cannula 530. Seal 550 defines an opening 552 in the central region of the seal. Therefore, surgical personnel will have the ability to maneuver the instruments within the cannula without compromising the integrity and effectiveness of seal 550.

A flexible zero-seal 540 for sealing cannula lumen 514 to prevent or minimize the loss of gasses through inlet opening 548 is provided within proximal housing portion 542. Flexible zero-seal 540 is designed to provide a positive seal whether or not an instrument is positioned therein. Flexible zero-seal 540 may be a flexible membrane having an expandable slit formed therein.

In use, a body incision or hole is typically made through the dermis of the patient with a trocar. Self-sealing cannula assembly 510 is then positioned through the body incision into an insufflated cavity. An instrument 600 is then inserted through opening 548, through zero-seal 540, into lumen 514, through the opening 552 in seal 550 and into the insufflated cavity.

During use of the instrument 600, the surgeon is required to maneuver the instrument 600 in a plurality of positions to complete the surgical procedure. Flexible cannula 530 is designed to accommodate at least a portion of the displacement caused by the instrument 600 being maneuvered by the surgeon. FIG. 6 illustrates the instrument 600 positioned within the cannula assembly 510 in the substantially vertical position. FIG. 7 illustrates the instrument 600 positioned within the cannula assembly 510 wherein the instrument 600 is displaced a distance x from the vertical axis at a location adjacent seal 550. The vertical dashed line in FIG. 7 represents the vertical axis through cannula assembly 510 and shows the displacement of instrument 600 from the vertical position. Due to the close proximity between seal 550 and instrument 600, as well as the connection between seal 550 and flexible cannula 530, as instrument 600 is displaced from the vertical axis, flexible cannula 530 is displaced a corresponding amount. As illustrated in FIG. 7, the distal end portion of instrument 600 is displaced to the left. The distal end portion 534 of flexible cannula 530 is also shifted to the left. As shown in the figure, the right side of the distal end portion 534 of flexible cannula 530 is shifted to the left, without compromising the integrity of seal 550.

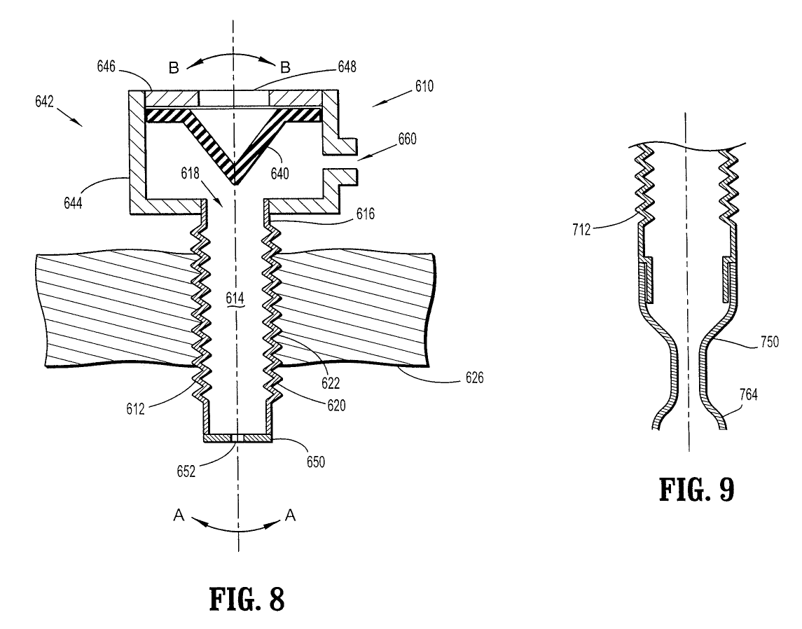

A flexible self-sealing cannula assembly in accordance with an embodiment of the present invention is shown generally as reference numeral 610 in FIG. 8 and includes a tubular portion 612 which defines a longitudinal lumen 614. The tubular portion includes a proximal end portion 616, which preferably defines an inlet opening 618, a central body portion 622, which is preferably cylindrical, and a distal end portion 620. Tubular portion 612 is preferably formed of flexible tubing. Tubular portion 612 is anchored in the dermis 626 of the patient, and the proximal and distal end portions 616 and 620, respectively, are free to move in the directions indicated by arrows A-A and B-B. Therefore, an instrument positioned within cannula assembly 610 may be maneuvered in the directions indicated by arrows A-A and B-B without compromising the integrity of seal 640 or instrument seal 650. The movement of tubular portion 612 is not limited to one particular axis, rather the movement is three-dimensional.

Self-sealing cannula assembly 610 also includes a proximal housing portion 642. Proximal housing portion 642 includes a cylindrical member 644 and a cover 646. The cylindrical member 644 defines an opening for receiving tubular portion 612. Proximal end portion 616 of the tubular portion 612 is secured in a manner that will form a seal between the proximal end portion 616 and cylindrical member 644. Cover 646 defines an inlet opening 648 for receiving surgical instruments into the self-sealing cannula assembly 610. Cover 646 engages the proximal end of cylindrical member 644 and may be attached thereto by means known to one having ordinary skill in the art. Although self-sealing cannula assembly 610 is illustrated as being formed of multiple components, e.g., proximal housing portion 642 is formed separately from the cannula body 612, it is envisioned that self-sealing cannula assembly 610, or any portion thereof, may be of monolithic construction.

An instrument seal 650 having a proximal surface and a distal surface is connected to the distal end portion 620 of the tubular portion 612. Instrument seal 650 defines an opening 652 in the central region of the seal. Since tubular portion 612 is flexible, surgical personnel will have the ability to maneuver the instruments within the cannula without compromising the effectiveness of seal 650.

Referring now to FIG. 9, a side cross-sectional view of a tubular portion 712 and instrument seal 750 of a flexible, self-sealing cannula assembly in accordance with another embodiment of the present disclosure is illustrated. In accordance with this embodiment, seal 750 is a tapered flexible seal. The distal end portion of seal 750 is shown having a flared end 764. Alternatively, the distal end portion of seal 750 may have a diameter which is equal to, or less than, the diameter of the tapered section.

With reference again to FIG. 8, a port 660 is formed in the proximal housing portion 642. Port 660 is used to introduce insufflation gas into the cavity of the patient. That is, with the cannula assembly 610 positioned within the dermis 626 of the patient, the insufflation gas flows into port 660, down through lumen 614, through opening 652 in instrument seal 650, and into the cavity of the patient. Once the cavity is sufficiently pressurized, a valve (not shown) connected to port 660 is closed to maintain the pressure within lumen 614 and the cavity of the patient. Thereafter, lumen 614 remains pressurized by the insufflation gas which enters into the lumen 614 through the opening 652 in seal 650. The pressure against the inside surface of the flexible tubular portion 612 maintains a seal between the outer surface of flexible tubular portion 612 and the inner surface of the dermis 626 of the patient. Therefore, the loss of insufflation gas around the circumference of the cannula assembly is effectively prevented.

It will be understood that various modifications may be made to the embodiments disclosed herein. For example, the cannula may have a variety of different shapes other than cylindrical, e.g., square, oval, rectangular, etc. Therefore, the above description should not be construed as limiting, but merely as exemplifications of preferred embodiments. Those skilled in the art will envision other modifications within the scope and spirit of the following claims.

What is claimed is:

1. A flexible cannula comprising:
a tubular member defining a lumen and having a proximal end portion and a distal end portion, wherein the tubular member is formed of a flexible material; and
a seal positioned adjacent the distal end portion of the tubular member wherein the seal defines an opening in a central portion thereof for receiving surgical instruments therethrough, and further wherein the seal seals the distal end portion of the lumen.

2. The flexible cannula as recited in claim 1 wherein the seal is a septum seal.

3. The flexible cannula as recited in claim 1 wherein the tubular member is formed of a flexible tubing.

4. The flexible cannula as recited in claim 1 wherein the cannula is substantially cylindrical in shape.

5. The flexible cannula as recited in claim 1 further comprising a housing positioned adjacent and proximal to the proximal end portion of the tubular member.

6. The flexible cannula as recited in claim 5 further comprising a seal mounted in the housing.

7. The flexible cannula as recited in claim 6 wherein the seal mounted in the housing is a zero-seal.

8. The flexible cannula as recited in claim 1 wherein the tubular member is connected to the housing at a proximal end of the cannula.

9. The flexible cannula as recited in claim 8 wherein a seal is formed at the connection between the tubular member and the housing.

10. The flexible cannula as recited in claim 1 wherein the seal is a tapered flexible seal.

11. The flexible cannula as recited in claim 1 further comprising a guide member attached to a distal end of the tubular member.

12. The flexible cannula as recited in claim 11 wherein the distal end of the guide member is beveled.

13. The flexible cannula as recited in claim 1 wherein the seal positioned adjacent the distal end portion of the tubular member is an instrument seal.

14. A self-sealing cannula assembly comprising:
a housing;
an elongate cannula body having a proximal end portion and a distal end portion wherein the cannula body is formed of a flexible material; and
an instrument seal mounted on the distal end portion of the elongate cannula body for sealing the distal end portion of the elongate cannula body.

15. The self-sealing cannula assembly as recited in claim 14 wherein the cannula body defines a longitudinal lumen.

16. The self-sealing cannula assembly as recited in claim 14 wherein the proximal end portion of the cannula body defines an inlet opening and the distal end portion defines an outlet opening.

17. The self-sealing cannula assembly as recited in claim 14 wherein the housing comprises a cylindrical member and a cover mounted on the cylindrical member.

18. The self-sealing cannula assembly as recited in claim 17 wherein the cover defines an inlet opening for receiving at least one surgical instrument into the self-sealing cannula assembly.

19. The self-sealing cannula assembly as recited in claim 14 further comprising a zero-seal mounted within the housing.

20. The self-sealing cannula assembly as recited in claim 14 wherein the instrument seal defines an opening in a central region of the instrument seal.

21. The self-sealing cannula assembly as recited in claim 14 wherein the seal is a tapered flexible seal.
